(19) United States
(12) Patent Application Publication
(10) Pub. No.: US 2008/0027751 A1
(43) Pub. Date: Jan. 31, 2008

(54) Medical product reservation, distribution and purchasing system and method

(76) Inventor: Marc Pappalardo, Boulder, CO (US)

Correspondence Address: JENNINGS, STROUSS & SALMON, P.L.C., 201 E. WASHINGTON ST., 11TH FLOOR, PHOENIX, AZ, 85004, US

(21) Appl. No.: 11/493,690

(22) Filed: Jul. 25, 2006

(51) Int. Cl. G06Q 10/00; A61B 5/00

(52) U.S. Cl. 705/2; 600/300

(57) ABSTRACT

A system and method for the reservation, distribution and purchasing of medical products is disclosed. More specifically, what is disclosed is a system and method of reserving and distributing pharmaceuticals, vaccines, medical devices and other medical supplies. The system and method disclosed herein is particularly applicable and useful in preparation for and during situations in which one or more medical products would be in short supply, such as during an epidemic, pandemic, or catastrophic events, such as natural disasters or man-made disasters.

### FIELD OF THE INVENTION

This invention relates generally to a system and method for distributing medical products. More specifically, this invention relates to a system and method of reserving and distributing pharmaceuticals, vaccines, medical devices and other medical supplies. This system and method is particularly useful and applicable to situations in which one or more medical products would be in short supply, such as during an epidemic, pandemic, or catastrophic events, such as natural disasters.

### BACKGROUND OF THE INVENTION

Presently, existing systems and methods of medical product supply and distribution have been unable to resolve and balance two major competing objectives: ensuring full and expeditious access to medical products, such as pharmaceuticals, vaccines and critical medical devices, when needed by the customer and avoiding the creation of oversupply when juxtaposed with actual demand in the marketplace.

Present attempts to resolve these two competing desires have been unsuccessful and have produced one of two results—both undesired. In some cases, for a variety of reasons production has not met demand, and a shortage of the needed medical product results, creating uncertainty, panic and potential for black market sales and/or potentially unsafe medical products being sold to customers. In other cases, attempts to avoid shortages have resulted in overproduction and excess supply, creating warehouses of aging product rapidly approaching its expiration date and/or excessive amounts of overhead, resulting in cash flow woes for the manufacturers and distributors of such medical products.

While some present systems and methods of supply prediction and production boast only moderate amounts of medical supply overproduction, even these moderate amounts result in unnecessary expense. For example, in the case of some pharmaceuticals that cost tens or hundreds of dollars per dose and medical devices or equipment, such as respirators, which cost tens or hundreds of thousands of dollars each, even a minimal excess supply or overproduction can be costly. Thus, no incentives exist in the marketplace to create stockpiles or surpluses of medical supplies that are not now in demand, or have a high probability of being in demand in the near future.

While the competing objectives of ensuring supply and reducing overproduction are global problems, resolution of the problem is particularly critical in market-driven, free-enterprise healthcare systems, such as in the United States. Presently, in the United States, manufacturers of medical products attempt to predict supply and demand through historical data, seasonal demand data, and perceived threat or risk. In the case of many medical products, such as lifesaving medical devices and pharmaceuticals, much of the production and supply created is based on the current demand without sufficient consideration for the potential harm that could be caused if an event, such as a catastrophe or pandemic, that has a relatively low probability of occurrence but a high potential for harm, actually occurs.

Therefore, in light of the constraints and limitations on present distributions systems, as well as the unreliability and uncertainty plaguing current distribution systems, coupled with the inability of the present distribution systems to accurately predict customer demand, a need exists for a system and method for providing customers with a reliable method of reserving, distributing and purchasing medical products such as pharmaceuticals and vital medical devices.

### BRIEF DESCRIPTION OF THE DRAWINGS

FIG. 1 is a flowchart illustrating the customer ordering and reservation process according to one embodiment of the present method.

FIG. 2 is a flowchart illustrating the distribution process according to one embodiment of the present invention.

### DETAILED DESCRIPTION OF VARIOUS EMBODIMENTS

This following disclosure provides for a system and method for distributing medical products. More specifically, the following discloses a system and method of reserving and distributing pharmaceuticals, vaccines, medical devices and other medical supplies. The system and method disclosed herein is particularly applicable and useful in preparation for and during situations in which one or more medical products would be in short supply, such as during an epidemic, pandemic, or catastrophic events, such as natural disasters or man-made disasters.

The disclosed system and method for reserving and distributing critical medical products, such as pharmaceuticals, medical devices and vaccines, is advantageous over any current system that presently exists in that the disclosed system and method allows for customers to reserve certain medical products in a manner that will allow them to have ready access to those medical products when and if the need arises.

The present system and method also provides for a more effective method of matching future customer demand for medical products with a ready supply and additionally provides a funding mechanism to support the generation of such a supply. Under the existing systems, either no or very meager monetary incentives exist for suppliers of medical products to create and manufacture a surplus supply when the demand in unpredictable or the probability of a demand does not reach a certain threshold.

Further, the disclosed system and method are advantageous over customers self-stockpiling for numerous reasons. First, although customers could arguably order and purchase the medical products themselves and stockpile them, no legitimate doctor should write a prescription for a particular medical product without an existing need for it. Second, the customer would likely have to replace the medical product every so many years, as most medical products have expiration dates. Third, in order for customers to get many medical products, especially pharmaceuticals, without a prescription, they must purchase them outside the United States, such as in Canada or Mexico, violating certain laws.

Thus, the disclosed system and method solves each of these problems by having a current, non-expired supply available here in the United States, ready and waiting for the customer to have an existing need and obtain the proper prescription (if applicable). Once the customer has a need and obtains any necessary prescription, the medical product is supplied without the fear of inadequate supply facing him.

According to the present disclosure, customers, including individuals, entities, or certain defined groups, such as medical practices, hospitals, insurance companies or other third party providers, access the disclosed medical product reservation, distribution and purchasing system (hereinafter also referred to as “the service” or “the system”) via one of a plurality of ways, and said service is capable of accepting said reservations for particular medical products, and providing reassurances, or even guarantees, that the customer will have access to a given medical product upon proper request for shipment of the medical product.

According to one embodiment of the present disclosure, the customer subscribes or enrolls in the service by providing certain key information, including personal identifying information and a request or reservation for a given medical product. Once the customer has reserved the medical product, the disclosed system and method employs one or more mechanisms to ensure that the particular reserved medical product is available upon customer request. In one embodiment, the availability of the medical product is guaranteed regardless of whether the medical product is generally in short supply or out of supply to the general population.

The service provided to the customer may employ one or more methods of acquiring access to or supply of medical products in order to ensure it can fill its orders upon customer request. In one embodiment, the service contracts directly with the manufacturer or supplier of the medical product, e.g., medication or device, and is contractually obligated to provide the service with a predetermined and/or contracted allotment of units of one or more medical products upon request by the service (hereinafter also referred to as “a supply contract”). According to the present disclosure, a supply contract includes, but is not limited to, an agreement to indirectly or directly supply the service (including third party contracts) or the service's customers, option agreements and purchase agreements. The contractual obligation to the service would be effective regardless of supply demands from other customers.

Under these supply contracts, the service would, in one embodiment, provide some form of consideration to the supplier or manufacturer. The consideration could and would likely create an incentive to increase the total supply of the medical product in the marketplace. Moreover, medical product manufacturers and suppliers could use these supply contracts and the data accumulated by the service as a more effective and efficient gauge for future demand for the medical product(s) at issue.

The same is true with the creation of a derivatives market. In a medical product derivatives market created hereunder, fluctuations in the price for a medical product in the derivatives market could assist the medical product manufacturer or supplier to more effectively and efficiently view trends in demands on a timelier basis and to use more reliable indicators.

Thus, either of the above alternate embodiments of the present invention have significant advantages over the current system. Namely, rather than predicting supply and demand through historical data, seasonal demand data, or some incomplete and tenuous analysis of perceived threat or risk, the present disclosure provides for a more effective and reliable method of determining supply and demand through present consumer data and demand. Thus, the present disclosure provides suppliers and manufacturers a method of tracking actual demand and then giving them a mechanism to alter their supply accordingly, as well as to provide alternate outlets for any excess supply.

Therefore, the suppliers and manufacturers can reduce the risk involved in under- or over-producing certain medical products, ultimately creating better incentives, namely market-based incentives, for suppliers and manufacturers of certain medical products to produce an adequate supply of certain needed or potentially needed pharmaceuticals, vaccines and medical devices that may otherwise be scarce or in short supply.

In an alternate embodiment, the service itself would maintain an adequate supply or stockpile of one or more medical products that have been reserved and may be requested at any point by the customer using the service. In this embodiment, the supply maintained by the service would ideally need to equal the total number of units of each medical product that could be requested by the customer. In an alternate embodiment, the service would employ a combination of stockpiling the medical product and entering into supply contracts.

However, except for products, such as vaccines, antidotes, antibiotics, and medical devices that would be needed for catastrophic events such as epidemics or pandemics, it is envisioned that an adequate supply could be maintained based on probability of use within a given time frame. For medical products most likely to be used in catastrophic events, epidemics and the like, it would be preferable to maintain a supply sufficient to provide all customers that subscribe to the service their reserved medical product in the event that every subscriber simultaneously requests its allotment.

In a further embodiment of this disclosure, the service would also serve as a pharmacy for subscribing customers in order to ensure that all of its stockpile's shelf life stays current and/or a distributor to other pharmacies or suppliers, by selling the stockpile with the shorter shelf life and keeping the newest medications in reserve. Having the ability to rotate potentially expiring inventory through a secondary outlet, such as a pharmacy, and in one embodiment, preferably a discount pharmacy, would solve problems involved with expiration of medical products that are in stockpile.

In one specific embodiment, the pharmacy is an Internet pharmacy. An Internet pharmacy has several advantages in that it allows the service disclosed herein to directly provide stockpiled medical products approaching expiration to customers without having to incur costs of involving a third party or having to engage in contracts with other pharmacies. Further, an Internet pharmacy would allow the stockpiled medical products to be available on a national basis, also increasing the service's ability to fully maximize the distribution of the medical products and avoid expiration, minimizing waste and destruction of the expired medical products.

According to the present disclosure, at least two potential pricing models exist for this service. In one embodiment, the customer would pay an initial reservation fee to reserve one or more medical products, which could be less than the cost of the medical product. Then, when the customer requests shipment of the medical product, the customer would pay an additional fee (with the total of all payments being at least equal to the cost of the medical product). In one embodiment, the cost of the medical product is preset at the time of reservation. In an alternate embodiment, the cost of the medical product is variable and the actual cost of the medical product is determined at the time the request for shipment is made, rather than at reservation.

Even if a customer had the methods and means to personally stockpile the medical product without needing the service, one embodiment of the present disclosure could allow the customer to ensure he will be able to meet his potential future needs for the medical product without initially having to pay upfront the cost of the product by, instead, paying the service an upfront fee for the reservation and paying the balance of the cost if and when the product is requested to be delivered.

In yet another embodiment, the service charges an annual fee to maintain the medical product on reserve for the customer. In either case, with a one-time fee or an annual fee, a further embodiment envisions that all or a portion of these fees could be applied to the cost of the medical product, whether the preset price or the market price at the time of shipment. However, in this embodiment, it is likely that the customer would then be charged a fee greater than the retail price upon dispensing of the medication in order to pay for the service's overhead in reserving and maintaining the supply of the medical product.

The particular alternative embodiment of the pricing model described above is especially applicable and viable when the service enters into contractual relationships with medical product manufacturers and/or suppliers, such as pharmacies, to guarantee sufficient supply to meet the customer demands. The above pricing model allows for the sale of options, futures, and/or other derivatives in the context of medical supplies.

The sale of futures would require the buyer to purchase a certain quantity of medical product at a given price on a certain date. However, sale of options would not obligate the buyer (here the service) to buy anything on any given date; yet the price would likely be predetermined. It is noted that the pricing model described above can be used regardless of how the supply of the medical products is secured or maintained.

The following alternate embodiment of a second pricing model is more applicable and viable when the service maintains the supply or stockpile itself rather than contracting out with medical product manufacturers and suppliers. However, it is also noted that this pricing model may be used in conjunction with that alternate embodiment as well and be incorporated in the contracted price and contractual provisions.

In this alternate pricing model, each customer is charged a one-time fee equal to at least the cost for the service (or in the alternative, the contracting medical product supplier) to purchase the reserved medical product, plus a recurring fee, used to maintain the reserved supply of the medical product. In yet another embodiment, the above-described system and method is also provided to health or other insurance companies, where they could offer this service to their insureds. In this embodiment, the service charges the insurance company a fee for administration of the system and method and provision of the above-described services.

By way of example only, a customer accesses the service (described below) and reserves Tamiflu®. The customer then pays the requisite fee for the reservation (as described above). When the customer believes that it may be in need of the Tamiflu®, the customer visits his physician to obtain a prescription for it. The service would only release the Tamiflu® to the customer upon the receipt of a valid prescription from a doctor (if a prescription is required). In a further embodiment, and as a value-added benefit, the service would offer its customers the ability to have one of a group of pre-selected doctors treat the customer, write any needed prescriptions and administer any necessary medications.

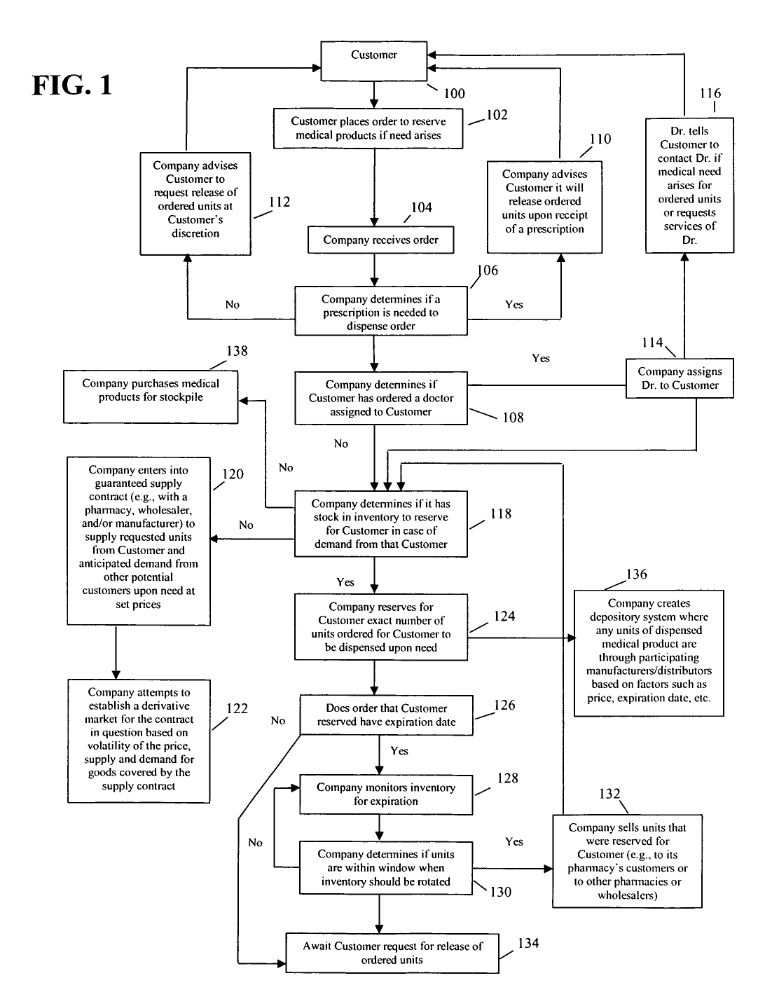

Turning now to FIG. 1, customer 100 accesses the disclosed medical product reservation, distribution and purchasing system. Access can be afforded via mail, telephone, email or an Internet web site among other methods known in the art.

Specifically in one embodiment, customer 100 is able to access the service home page, also referred to herein as the medical product reservation home page, of the host web site through accessing the medical product reservation service URL (Uniform Resource Locator). URL access may occur by numerous and varied methods. By way of example, the customer 100 may type the desired URL directly into the browser. In addition, the customer 100 may access the URL by selecting a hyperlink displayed on another web site or web page that links the customer 100 computer to the home page.

Hyperlinks displayed on a web page transfer the customer 100 to a different web site or web page when selected by the customer 100. The hyperlink to the medical product reservation home page may be included as part of an e-mail message displayed by the customer 100's computer. The customer 100 can also search the Internet for the medical product reservation web site using key terms in any Internet search engine, such as Google. In any of these examples, when the customer 100 selects the hyperlink, the customer 100 computer is directed to the home page. The home page is then displayed on customer 100's computer screen. Once the customer 100 has the home page displayed, the customer 100 can begin setting up an account and placing orders or reservations as described below.

If the customer 100 does not want to use the Internet to access the medical product reservation system, the customer 100 completes a hand-written paper request, and submits it, for example via mail or facsimile, or calls via the telephone a designated number, such as a 1-800 number. The particular methods of effectuating data entry for each of these modes of customer 100 orders or reservations are known in the art and all accomplish the same end of setting up a customer account and reserving desired medical products for the customer, including effectuating a payment method.

In order to provide the necessary security, in the case of HIPAA-compliance requirements, or other adequate security relating to the storage and use of the customer's personal information, including medical data, financial data and other individual identifying information, the present disclosure envisions and anticipates using secure techniques known in the art for electronic transmission of data. Accordingly, the system and method disclosed herein will incorporate where appropriate the necessary data-encryption protocols, firewalls or other security features, either as mandated by applicable law, or as deemed necessary for protecting and securing the privacy of the customer's records.

Once customer 100 accesses the system, customer 100 creates an account within the system. The customer creates a new account by selecting new account setup and following the instructions on how to create a new account. For a new account, the customer may be required to enter data, such as name, address, e-mail, and telephone number. Additional information may be required, including, but not limited to, billing data, and billing contact information. In other embodiments, some medical data may also be required, for example, physician name or medical history, insurance company, or other personal information necessary to properly determine the correct medical product selection for the customer.

Then, in step 102, the customer places an order to reserve one or more medical products. In addition the customer is asked to provide the number of units of the requested product. In one embodiment, the system provides education and guidance for the customer to assist him in determining the number of units to order based upon potential need and dispensing requirements for the medical product. For example, if one dose of vaccine is sufficient for immunization, the system would advise the customer to order one unit per person. However, if the customer is reserving penicillin and an average prescription of penicillin is thirty doses, then the customer would be advised to order thirty units per person.

As discussed above, medical products include, but are not limited to, pharmaceuticals, vaccines, and medical devices. In step 104, the company receives the order from the customer and in step 106, determines if a prescription is needed in order to dispense the order at the time the customer makes the eventual request for shipment.

If a prescription is required, the system (also referred to herein as “the company” for ease of reference to the entity administering the disclosed system and method) advises the customer in step 110 that it will release the ordered medical product only upon receipt of a valid prescription. If a prescription is not required, the system advises the customer in step 112 that the ordered medical product will be released upon request, at the customer's discretion, without any further documentation needed.

In an alternate embodiment of the disclosed system and method, the system is capable of providing a physician referral or assigning a physician to a particular customer. If a customer desires a physician referral or to be assigned a physician, the customer responds affirmatively to a prompt asking if the customer would like a doctor. In step 108, the system determines if the customer has requested a physician be assigned to the customer.

If the customer answers in the affirmative, in step 114, the customer is assigned a physician by the system. The primary goal of the physician assignment and referral aspects of the present disclosure is to provide the customer with a physician whom the customer can contact if the customer believes the need has arisen to receive the medical product. Once the customer has requested a physician, the system matches a physician with the customer based on predetermined criteria such as, for example, medical product requested, customer location and/or customer insurance information. In step 116, the system notifies both the physician and the customer of the physician assignment, and either the system or the physician relays information regarding how and when to contact the physician if and when the customer believes the medical need has arisen for the ordered medical product(s). The customer may also contact the physician at any time to request the services of the physician.

If the customer declines physician referral or assignment, the system proceeds to step 118 and determines if the inventory of the ordered medical product is available for the customer if and when the customer makes a request for shipment of the ordered medical product. If the inventory is inadequate, the system then either (1) orders more inventory to increase its own stock or supply of the particular medical product, or (2) as illustrated in step 120, enters into a guaranteed supply contract to supply the requested medical product.

In step 120, the guaranteed supply contract could be with a pharmacy, wholesaler, manufacturer or other supplier of the medical product. The guaranteed supply contract would provide that the pharmacy, wholesaler, manufacturer or other supplier would provide the requested units of medical product upon demand. The contract would further provide that the supply of the medical products would be provided at set, predetermined prices and would be guaranteed regardless of market demand or shortages elsewhere.

In step 122, the system would then attempt to establish an options, futures and/or other derivatives market for these supply contracts. As with other derivative markets, the derivative market established according to the present disclosure would be established based on volatility of the price as well as supply and demand for the medical product(s) covered by the supply contract. For example, a derivatives market for supply contracts for Tamiflu® may experience an increase in the price of that security in the event that a case of avian flu was discovered in the United States.

Prior to the present disclosed system and method, there have been several barriers to the creation and existence of a derivatives market for medical products. First, most manufacturers and suppliers are unwilling to take the financial risk of carrying a supply for future demand beyond a conservative analysis of short-term estimated demand. Primarily this has been a barrier because of the factor stated above, namely the lack of an effective and reliable method to measure future demand that is based on actual market demand rather than less reliable historical data and risk data. Second, in the medical products markets, especially in the area of vaccines and pharmaceuticals, there are a limited number of manufacturers and suppliers for a certain product. Third, the regulatory burdens are more extensive than most other markets, for example, FDA, DEA, CLIA, state and local rules governing prescriptions, pharmacies, and drug distribution, licensure laws, etc., are all implicated in this type of market.

However, the present disclosure provides a mechanism that gives the manufacturer or supplier insight as to when to increase production or request additional supply. Creation of a market, such as a futures market, provides a reliable sign of demand and gives the manufacturer the time to increase production in time to meet that demand. For example, when the price of the future increases, the manufacturer will understand that this means the market has detected an actual increase in future demand, and thus this can assist the manufacturer in planning to meet this future demand-before it becomes a present demand and the supply is inadequate. This is particularly important in markets such as pharmaceutical and vaccine markets where increasing production and supply takes time and planning.

In step 124, if the system identifies that an adequate supply exists and/or that a supply contract exists, the system reserves for the customer the number of units requested. Next, in step 126, the system assesses whether the medical product reserved by the customer is subject to expiration and determines what the expiration date is. The expiration date issue is less of a concern, and this step may even be eliminated in cases where the medical product is being supplied through a supply contract directly to the customer by a manufacturer, supplier, pharmacy or other contracted third party who is responsible for monitoring expiration and delivering only viable (unexpired) medical products upon customer request.

However, if the product is being stockpiled, stored or otherwise monitored internally by the system, the system monitors the inventory for expiration in step 128. In step 130, at a predetermined point relative to the expiration date, the system alerts that the inventory should be rotated. In rotation of inventory, as shown in step 132, the system sells to other outlets, including to pharmacies, wholesalers, distributors or other markets where the inventory will sell before expiration, those units that are approaching expiration.

In the event that a customer attempts to place an order for a medical product and the system does not have an adequate supply of the medical product in question to meet all or a portion of the order, the system can place that portion of the order that it can fill, suggest an alternative medical product for all or a portion of the requested order and allow an order for the alternative medical product to be filled, attempt to fill the order through other outlets, direct the customer to other potential sources that have some form of referral fee or revenue-sharing arrangement with the system, and/or allow the customer to elect to be notified in the event that the system is later able to meet any unfilled portion of the customer's order.

In an alternate embodiment, the system also includes a pharmacy and/or medical product distributor, to sell units approaching expiration. Thus, the inventory can be internally rotated to the pharmacy and/or distributor for sale, and new stock can replace it, awaiting customer requests. In one version of this embodiment, the system automatically determines when the inventory should be transferred from the customer stock (awaiting request from the customer) to the pharmacy. In this manner, a smooth internal rotation of medical products subject to expiration can occur and the majority of medical product is not wasted or subject to destruction.

In an alternate embodiment, a customer could, at the time of order or delivery, select different pricing models for the medical product in question that is based on how near or how far the expiration date is to the date of delivery. According to the present disclosure as it relates to customers of the system, a customer's price for the ordered product could fluctuate based on whether the customer requested delivery of product with an expiration date that is relatively close in time to the delivery date versus a request for delivery of the same medical product with a relatively long expiration date.

As shown in step 136, the system with one or more manufacturers or suppliers could collectively serve as a form of bank or depository system that would reduce the loss of medical product due to expiration, foster better purchase terms for participating members by allowing them to opt for delivery under a supply contract of other participating members instead of their own supply contracts or stockpile, and/or allow the pricing of the product to be based on how near or how far the expiration date is relative to the date of delivery.

For example, a participating member, including the system, could request delivery of a medical product in question from the stockpile of another participating member and/or by or through another participating member's supply contract, with that other participating member receiving a credit in the system which could be similar to inter-bank debits and credits under the Federal Reserve System, as disclosed in prior art.

For example, participating member “A” could select to utilize the stockpile of participating member “B” to fill a customer order because that customer was willing to accept a relatively short expiration date, and participating member B would then have a corresponding right to agreed-upon medical products in participating member A's stockpile or supply contract, which may also involve the exchange of consideration between the participating members based on factors such as the purchase price, product expiration date, term of the supply contracts in question used to fill the order or consummate the inter-participating members' transaction.

Additionally, the system could set up a national registry or repository where medical products near expiration could be auctioned to qualified purchasers. Such a registry could take the form of a limited-access Internet auction site. Further, the medical products near expiration could be donated to federal government agencies or other charities that provide medical care, pharmaceuticals, vaccines and other medical products to underprivileged and/or indigent patients and/or shipped to foreign countries, namely third world countries in order to maximize the use of the medical products without having to destroy them upon expiration. Certain tax incentives and other forms of compensation could be available to help defray the costs of the donated medical products.

If the medical product does not have an expiration date in step 126, the medical product is stored and awaits the customer's request for release of the ordered units in step 134. Further, the medical product which has not been rotated to sell based on expiration date also awaits the customer's request for release of the ordered units, also in step 134.

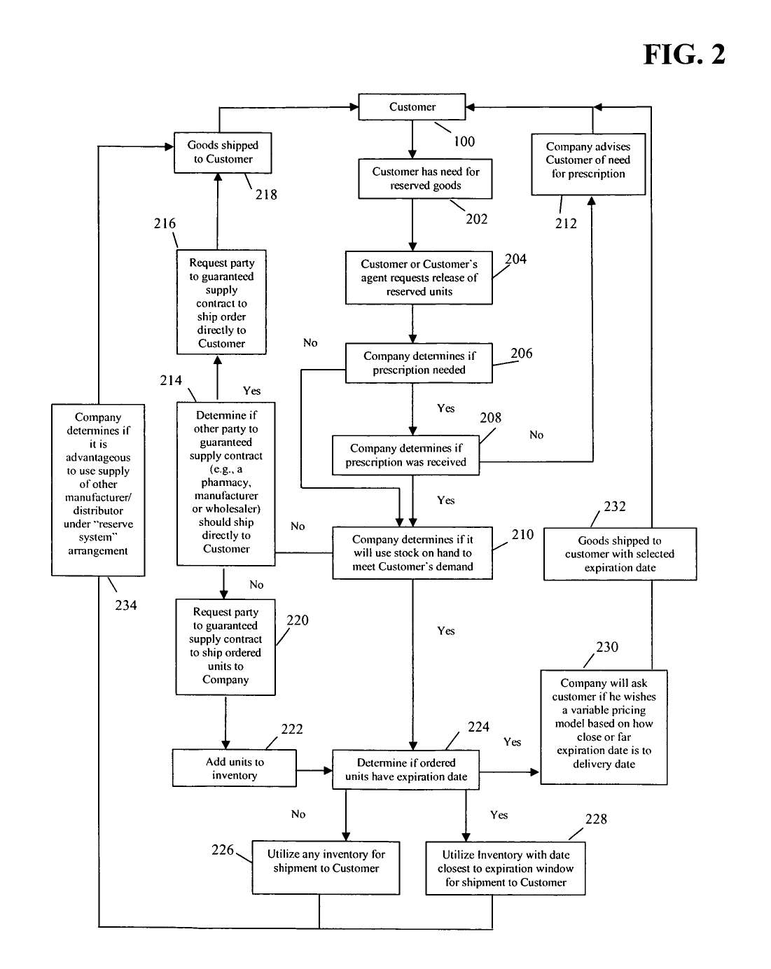

Now turning to FIG. 2, customer 100 determines in step 202, either on his own or through visit to a physician, that he has a need for one or more medical products that he has ordered and reserved through the system. In step 204, the customer or customer's agent requests release of the desired number of reserved units and requests shipment to the customer. In step 206, the system determines if a prescription is needed to release and ship the desired medical product. If so, in step 208, the system determines if a prescription has already been received with the request for release and shipment.

If the system determines no prescription has been received, the system then contacts the customer, and/or any assigned physician in step 212, and advises of the need to receive a valid prescription for the requested medical product prior to the system's being able to release and ship the medical product to the customer. Once a prescription is received, or if one already exists on the system, in step 210, the system determines if the stock on hand (the internal inventory) will meet customer's demand. The internal inventory closest to expiration will be shipped first.

If not, the system determines if another party is contracted to guarantee supply in step 214. If so, the system then instructs that party, e.g., the pharmacy, manufacturer, wholesaler, or other supplier, to either ship the medical product directly to the customer (steps 216-218) or to supply the medical product to the system to add to its inventory and/or ship to the customer (steps 220-222). Specifically, in step 216, the system requests the contracting party to ship the requested medical product, preferably directly to the customer. Thus, in step 218, the requested medical product is shipped to the customer.

If in steps 220 and 222, the contracting party guaranteeing the supply ships medical products to add to the system's internal inventory, the system then determines, upon receipt, in step 224, whether or not the units have an expiration date. If so, the internal inventory closest to expiration will be shipped first, as shown in step 228.

In an alternate embodiment, in step 230, the system will ask the customer if he wishes to utilize a variable pricing model based on how close or how far the expiration date is to the delivery date. In this alternate embodiment, if a customer is willing to accept inventory closer to expiration, the price of that will be reduced accordingly. If the customer opts for this variable pricing model, the units with the selected expiration date will be shipped to the customer in step 232.

If the medical product at issue has no expiration date, the system does not distinguish between inventory for shipment, and in step 226, any inventory is appropriate for shipment to the customer, and in step 218, the requested medical product is released and shipped to the customer for use. In an alternate embodiment, as shown in step 234, the system, following resolution of the expiration date issues, determines if it is advantageous to use the supply of a third party under the “reserve system” arrangement. If so, the system invokes its contract with the third party, and either has the third party supply directly or indirectly the units to the customer.

In the event a contract cannot be reached with an appropriate medical product manufacturer or supplier in the United States, and the service cannot itself acquire the medical product in the United States, the service would explore other options. For example, currently, Tamiflu® cannot be purchased in the United States by private entities or individuals at the request of the United States government due to shortages in the United States. However, Tamiflu® can be purchased in Canada because Canada has a sufficient stockpile to treat its people. Thus, the service would explore options, from an operations standpoint, as operating a Canadian subsidiary as part of the service.

While at a superficial level, the disclosed system and method may appear at odds with public policy considerations (in that it may appear that those from higher socio-economic classes will get medications at the expense of those from lower ones), this is not the case. Rather, the disclosed system and method basically privatizes (as with most healthcare functions in the United States) an obligation to maintain an adequate supply of needed medical products that the government or any other existing system and method has not yet accomplished.

For example, with respect to Tamiflu®, the government is purposefully not stockpiling sufficient dosages in order to ensure that the general population will be able to receive medications in the event of a pandemic, where other countries, like Canada, have taken on such a duty and expense. The disclosed system and method essentially supplements the supply that the government has agreed to purchase for its people, thereby increasing the number of individual in the United States that have real access to medical products and resulting treatments.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventor that the words and phrases in the specification and claims be given the ordinary and accustomed meanings to those of ordinary skill in the applicable art(s).

The foregoing description of a preferred embodiment and best mode of the invention known to the applicant at this time of filing the application has been presented and is intended for the purposes of illustration and description. It is not intended to be exhaustive or limit the invention to the precise form disclosed and many modifications and variations are possible in the light of the above teachings. The embodiment was chosen and described in order to best explain the principles of the invention and its practical application and to enable others skilled in the art to best utilize the invention in various embodiments and with various modifications as are suited to the particular use contemplated. Therefore, it is intended that the invention not be limited to the particular embodiments disclosed for carrying out the invention.

What is claimed is:

1. A method for providing a customer substantially guaranteed future access to a medical product upon demand, comprising:
providing to a customer an array of possible medical products, wherein the customer is capable of selecting a medical product from the array;
allowing the customer to place an order for the selected medical product, wherein the customer does not know at the time the order is placed when the customer will need the medical product;
determining whether a prescription is required to dispense the medical product to the customer;
verifying that the medical product will likely be available to the customer upon future request; and
reserving the medical product for the customer upon receipt of the order and verification of the availability of the medical product.

2. The method of claim 1, further including notifying the customer if a prescription is required.

3. The method of claim 1, further including notifying the customer's physician if a prescription is required.

4. The method of claim 1, further including providing the customer with the option of requesting a physician.

5. The method of claim 4, further including assigning a physician to the customer upon request.

6. The method of claim 1, further including charging a fee when the customer places the order.

7. The method of claim 1, further including receiving a request from the customer to dispense the medical product to the customer.

8. The method of claim 7, further including determining at the time the request is received whether a prescription is required to dispense the medical product to the customer.

9. The method of claim 8, further including verifying that the customer has received a prescription for the medical product.

10. The method of claim 8, further including dispensing the medical product to the customer if no prescription is required.

11. The method of claim 9, further including dispensing the medical product to the customer upon verification that the customer has received a prescription.

12. The method of claim 7, further including charging a fee when the customer requests that the medical product be dispensed.

13. The method of claim 7, further including charging the customer a fee periodically between the time the customer places the order and the time the customer requests that the medical product be dispensed.

14. A method for providing a customer substantially guaranteed future access to a medical product upon demand, comprising:
providing to a customer an array of possible medical products, wherein the customer is capable of selecting a medical product from the array;
allowing the customer to place an order for the selected medical product, wherein the customer does not know at the time the order is placed if the customer will need the medical product;
determining whether a prescription is required to dispense the medical product to the customer;
verifying that the medical product will likely be available to the customer upon future request; and
reserving the medical product for the customer upon receipt of the order and verification of the availability of the medical product.

15. A method for providing a customer substantially guaranteed future access to a medical product upon demand, comprising:
providing to a customer an array of possible medical products, wherein the customer is capable of selecting a medical product from the array;
allowing the customer to place an order for the selected medical product, wherein the customer does not know at the time the order is placed when or if the customer will need the medical product;
determining whether a prescription is required to dispense the medical product to the customer;
verifying that the medical product will likely be available to the customer upon future request; and
reserving the medical product for the customer upon receipt of the order and verification of the availability of the medical product.

16. A method of substantially guaranteeing the availability of a medical product upon future request from a customer, comprising:
providing to a customer an array of possible medical products, wherein the customer is capable of selecting a medical product from the array;
allowing the customer to place an order for the selected medical product, wherein the customer does not know at the time the order is placed when the customer will need the medical product;
determining the availability of the medical product, wherein the availability is determined first by evaluating internal inventory and second by evaluating existing supply contracts; and
contracting with another supplier if the determination is made that the medical product will likely not be available to the customer upon future request.

17. The method of claim 16, further including establishing a market for the supply contracts.

18. The method of claim 17, wherein the market is selected from a group consisting of: an option market; a futures market; and a derivatives market.

19. The method of claim 17, wherein the market is based on an independent factor, the independent factor being selected from a group consisting of: the volatility of the price of the medical product; the supply of the medical product; and the demand for the medical product.

20. A method of substantially guaranteeing the availability of an unexpired medical product upon future request from a customer, comprising:
providing to a customer an array of possible medical products, wherein the customer is capable of selecting a medical product from the array;
allowing the customer to place an order for the selected medical product, wherein the customer does not know at the time the order is placed when the customer will need the medical product;
determining that the ordered medical product exists in internal inventory, wherein the expiration date of the medical product has been recorded;
monitoring the expiration date of the medical product; and
rotating the medical product out of inventory at a predetermined time prior to the expiration date.

21. The method of claim 20, further including:
adding the ordered medical product to the internal inventory, wherein a determination is made that the medical product does not exist in internal inventory;
determining the expiration date of the added medical product; and
recording the expiration date of the added medical product.

22. The method of claim 20, wherein the inventory is rotated to a third party.

23. The method of claim 22, wherein the third party is selected from a group consisting of: a pharmacy, a distributor, a supplier, a manufacturer, a government agency, a charitable organization, a foreign country, a repository, a reserve, a clearinghouse, an auction site, and an individual.

24. The method of claim 23, wherein the pharmacy is operated via the Internet.

25. The method of claim 22, wherein the inventory is sold to the third party.

26. The method of claim 25, wherein the sale price of the inventory is variable depending on the amount of time between the sale date and the expiration date.

27. A method of predicting future demand for a medical product, comprising:
contracting with a supplier for a medical product; and
creating a market for the contract, wherein the market is selected from a group consisting of: a futures market; an options market; and a derivatives market.

28. The method of claim 27, wherein the contract is bundled with a plurality of contracts.

29. The method of claim 27, wherein the market is based on an independent factor, the independent factor being selected from a group consisting of: the volatility of the price of the medical product; the supply of the medical product; and the demand for the medical product.

30. The method of claim 27, wherein the sale price of the medical product is variable depending on the amount of time between the sale date and the expiration date

31. A computer-readable storage medium containing computer executable code for instructing a server computer to perform the steps of:
providing to a customer an array of possible medical products, wherein the customer is capable of selecting a medical product from the array;
allowing the customer to place an order for the selected medical product, wherein the customer does not know at the time the order is placed when the customer will need the medical product;
determining whether a prescription is required to dispense the medical product to the customer;
verifying that the medical product will likely be available to the customer upon future request; and
reserving the medical product for the customer upon receipt of the order and verification of the availability of the medical product.

32. A system for providing a customer substantially guaranteed future access to a medical product upon demand, comprising:
a host server computer including a web site having one or more content-based web pages and in communication with one or more distribution mediums, wherein, in response to a communication from a recipient computer, said host server provides said recipient computer an array of possible medical products, wherein said recipient computer is operable by a customer and wherein said recipient computer is capable of selecting a medical product from the array and placing an order for said medical product, wherein the customer does not know at the time the order is placed when the customer will need the medical product; and wherein said host server verifies that the medical product will likely be available to the customer upon future request; and reserves the medical product for the customer upon receipt of the order and verification of the availability of the medical product.

33. A system for substantially guaranteeing the availability of a medical product upon future request from a customer, comprising:
a first medical product supplier contracting with a second medical product supplier for the supply of a medical product, the contract resulting in a desired outcome, wherein the desired outcome is selected from a group consisting of: the first medical product supplier reducing the number of expiring medical products in its inventory; varying the sale price of the medical product depending on the amount of time between the sale date and the expiration date; and delivery of a medical product to a customer of the first medical product supplier under a supply contract of the second medical product supplier.

34. The system of claim 33, wherein the medical product supplier is selected from a group consisting of: a pharmacy, a distributor, a supplier, a manufacturer, a government agency, a charitable organization, a foreign country, a repository, a reserve, a clearinghouse, an auction site, and an individual.

35. The system of claim 33, further including issuing a credit to the second medical product supplier when the first medical product supplier delivers a medical product to a customer under the second medical product supplier's supply contract.

36. The system of claim 35, further including issuing a debit to the first medical product supplier when the first medical product supplier delivers a medical product to a customer under the second medical product supplier's supply contract.

37. The system of claim 36, wherein the credits and debits are issued in a manner substantially similar to inter-bank credits and debits under the Federal Reserve system.

38. A system for distributing medical products prior to expiration of the medical product, comprising:
a centralized location for the listing of available medical products from a plurality of medical product suppliers, the centralized location being capable of effectuating the distribution of the medical products to one or more third parties, wherein the desired outcome of the distribution is to reduce the number of medical products reaching expiration.

39. The system of claim 38, wherein the centralized location is selected from a group consisting of: an international registry, a national registry, a geographic registry, an international repository, a national repository, a geographic repository, a depository, a clearinghouse, an auction house, an Internet site, an Internet auction site, a government agency, a private entity, a non-profit organization, and an individual.

40. The system of claim 38, wherein the third party is selected from a group consisting of: a pharmacy, a distributor, a supplier, a manufacturer, a government agency, a charitable organization, a foreign country, a repository, a reserve, a clearinghouse, an auction site and an individual.

41. The system of claim 38, wherein the medical products are donated by the medical product supplier.

42. The system of claim 38, wherein the distribution of the medical products is pursuant to a purchase contract, the purchase contract reflecting a varying sale price of the medical product depending on the amount of time between the sale date and the expiration date.

43. The system of claim 38, wherein the medical products are distributed to the highest bidder in an auction of the medical products.
